(19) United States
(12) Patent Application Publication
(10) Pub. No.: US 2016/0011105 A1
(43) Pub. Date: Jan. 14, 2016

(54) Light Extinction Tomography for Measurement of Ice Crystals and Other Small Particles

(71) Applicants: Timothy J. Bencic, Highland Heights, OH (US); David P. Rohler, Shaker Heights, OH (US); Amy F. Fagan, Fairview Park, OH (US); Steven H. Izen, Shaker Heights, OH (US); Arjun K. Maniyedath, Beachwood, OH (US)

(72) Inventors: Timothy J. Bencic, Highland Heights, OH (US); David P. Rohler, Shaker Heights, OH (US); Amy F. Fagan, Fairview Park, OH (US); Steven H. Izen, Shaker Heights, OH (US); Arjun K. Maniyedath, Beachwood, OH (US)

(21) Appl. No.: 14/751,085

(22) Filed: Jun. 25, 2015

Related U.S. Application Data
(60) Provisional application No. 62/017,143, filed on Jun. 25, 2014.

(51) Int. Cl. G01N 21/47; G01N 33/00

(52) U.S. Cl. CPC G01N 21/47; G01N 33/0009

(57) ABSTRACT

Systems and methods for imaging and detection of small liquid and solid water particles in different spray conditions includes visible light laser diodes that are pulsed across the area of interest and optical detectors that measure the extinction of light intensity at different directions. The attenuated light projections across the field of view are reconstructed to yield an image of the particles that crossed the plane of light. A wind tunnel is a major tool used in understanding of ice formation and the performance of aircraft engine components. The measurement of the spray provides calibration and, to date, wind tunnel calibration has been time consuming and expensive. This system and method provide near real-time in-situ quasi-quantitative full-field ice/water content data and the corresponding reconstructed images for analysis. The support frame, source-detector configurations, acquisition, simulation, and reconstruction methods of the light emission tomography technology are also disclosed.

### CROSS-REFERENCE TO RELATED APPLICATIONS

This application claims priority to U.S. Provisional Patent Application No. 62/017,143 filed Jun. 25, 2014, incorporated herein by reference.

### STATEMENT REGARDING FEDERALLY SPONSORED RESEARCH OR DEVELOPMENT

The work underlying this patent was developed under contract # NNC11CA25C with NASA/Glenn Research Center. Government rights to this invention are defined by FAR 52.227-11.

### BACKGROUND OF THE INVENTION

There have been over 200 documented cases of jet engine power loss events during flight at high altitudes due to ingestion of ice particles. The events typically occur at altitudes above 22,000 feet and near deep convective systems, often in tropical regions. It is recognized in the industry that super cooled liquid water does not exist in large quantities at these high altitudes and therefore it is expected that the events are due to the ingestion of ice particles.

Based on this recent interest in ice particle threat to engines in flight, the NASA Glenn Research Center (GRC) installed the capability to produce ice crystal and mixed phase water clouds in the Propulsion Systems Laboratory (PSL) Test Cell 3. The ice crystal cloud operational parameters, developed with input from industry, were Median Volumetric Diameter (MVD) from 40 to 60 μm and Total Water Content (TWC) from 0.5 to 9.0 g/m3. PSL is currently the only engine test facility that can simulate both altitude effects and an ice crystal cloud. It is a continuous flow facility that creates the temperature and pressure inlet conditions that propulsion systems experience in high-speed, high-altitude flight. Specifically for the icing system, the total temperature can be controlled between +45 to −60° F., pressure altitude from 4,000 to 40,000 feet (facility limit is 90,000 feet), and Mach from 0.15 to 0.8 (facility limit is Mach 3.0).

Within this facility, there was a specific need to develop a non-intrusive system to measure the conditions of a cloud that enters an aircraft engine in the PSL. The system must (1) have the capability to be operated remotely, (2) have minimal optical access, (3) no moving parts, (4) fast acquisition and (5) good resolution in a pipe that can structurally support an aircraft engine in close proximity.

An earlier study of this problem is described in REF. 1.

### BRIEF SUMMARY OF THE INVENTION

The invention is a light extinction tomography system for use in detecting small liquid and solid (ice) water particles in different spray conditions. Visible light laser diodes are pulsed across the area of interest and the extinction or loss of light intensity is measured at many different directions. The attenuated light projections across the field of view can be reconstructed to yield an image of the particles that crossed the plane of light. This is very similar to Computed Tomography (CT) in the medical imaging field in which slices of density through the body can generate images in the interior.

These and other aspects of the disclosure and related inventions are further described herein with reference to the accompanying figures.

### DESCRIPTION OF THE DRAWINGS

FIG. 1 is a schematic drawing of a wind tunnel depicting the location of the tomography duct relative to the spray bars and engine to be tested.

FIG. 2 is a photograph of the light extinction tomography prototype installed in the NASA/Glenn Propulsion Systems Lab (PSL) at the tomography duct pipe exit.

FIG. 3 is a photograph of the light extinction tomography prototype installed in the PSL specifically showing the cabling and installation of the laser sources and optical detectors.

FIG. 4 is a schematic illustration of a rectangular icing research tunnel in which the inner circle designates the region for which accurate imaging is desired and the small dots represent candidate positions of source and detectors.

FIG. 5 is a schematic illustration of the optical path for one laser source and the detectors that can receive useful signals from that source.

FIG. 6 illustrates a use of the Simulator in which the upper left image shows an ideal cross image, the upper right image shows an ideal circles image and images in the lower left and lower right show the result of reconstructing the cross image and circles image, respectively, using one embodiment of our reconstruction algorithm.

FIG. 7 shows characterization of the detection system utilizing fiber coupling (right image) from the tomography spool to the camera (upper left) to make the light intensity measurements for which a sample data image (lower left) shows the light collected by 120 fibers.

FIG. 8 demonstrates the projections for several sources to detectors coverage in the left image; a phantom image (center) is used for a resolution study to demonstrate the resolution quality between the center of the ring and the outer regions near the wall of the ring (right image).

FIG. 9 is a photograph illustrating the use of a prototype tomography ring for measuring the density of a water spray.

### DETAILED DESCRIPTION OF THE INVENTION

The invention is an optical tomography system to be used in particle density detection. The primary application considered here is the measurement of ice or water particle density in a cross section of a flow through a wind tunnel, though there are other applications of the invention as detailed below.

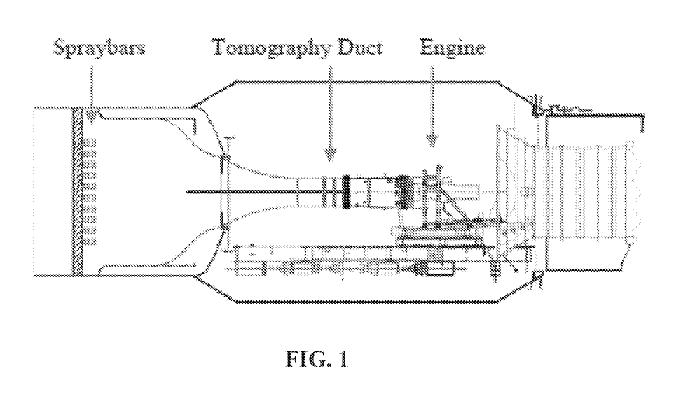

In one embodiment, the invention is designed to be integrated into the walls of a wind tunnel (FIG. 1) so as not to impede or interfere with the flow being measured.

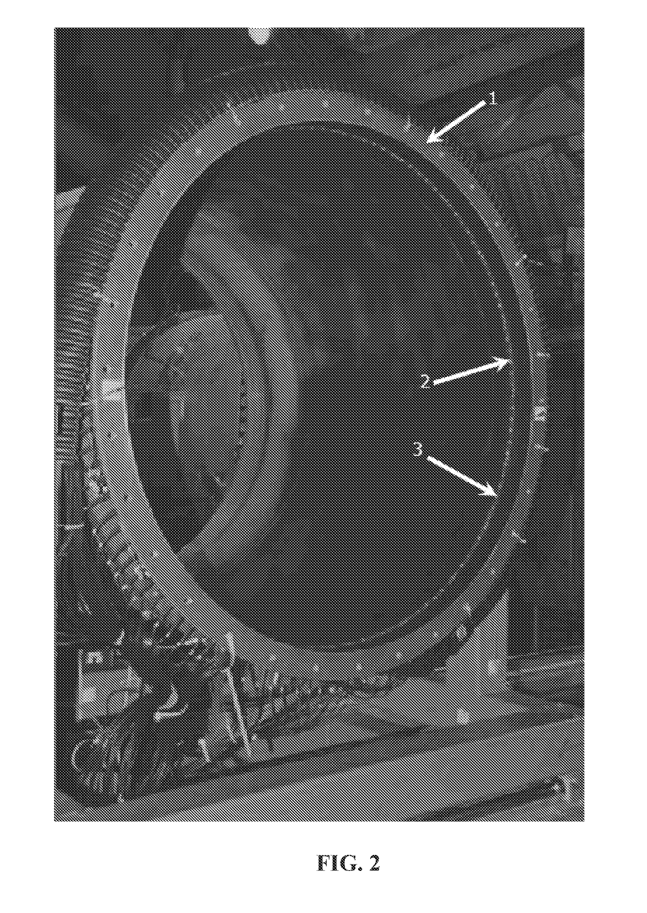

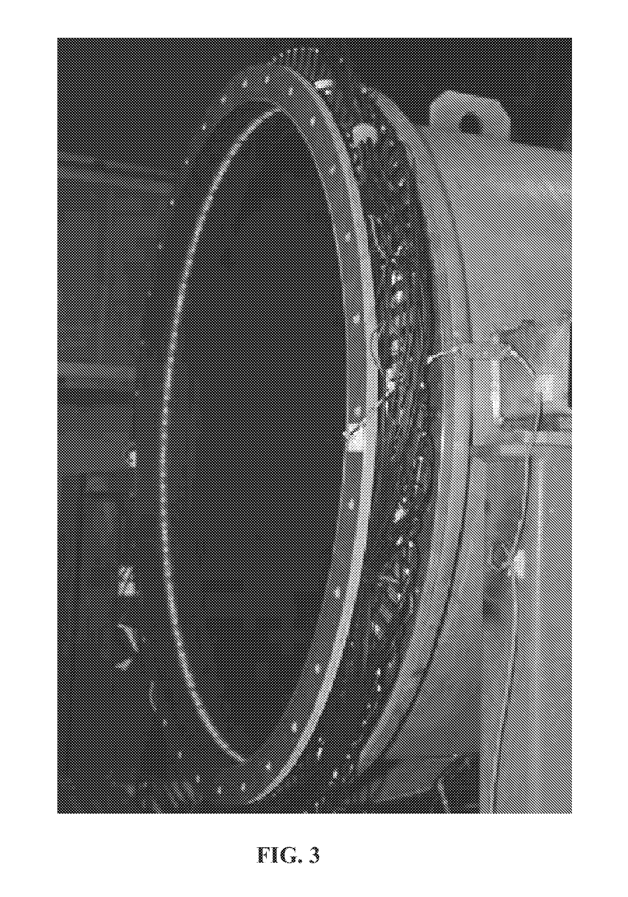

The device, shown in FIG. 2 and FIG. 3, includes (1) a support frame, (2) a collection of light sources distributed about the support frame, (3) a collection of optical detectors distributed about the support frame. In addition to the components shown in these figures, the device includes (4) a control system to illuminate the light sources in the desired illumination pattern, (5) a detection system to collect the measurements from the optical detectors, (6) a processing system to reconstruct an extinction map from the measurements on the detectors and (7) a control system to provide a user interface and coordinate the illumination and measurement functions.

### Support Frame

The support frame is nominally circular to conform to the wall of the wind tunnel in which the frame is to be mounted. In some embodiments the frame becomes part of the wall of the tunnel. In other embodiments, the frame can be inserted into the wind tunnel and mounted within the wind tunnel. The invention can work in any size wind tunnel provided the frame is scaled to the size of the wind tunnel.

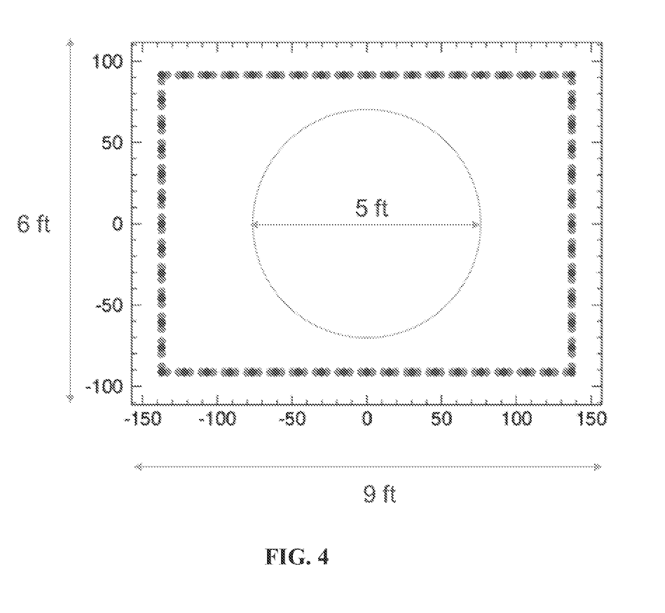

In other embodiments, the frame can have non-circular cross-sections. For example, if the invention were to be used in the NASA-Glenn Icing Research Tunnel, the frame would have a rectangular cross-section similar to that shown in FIG. 4.

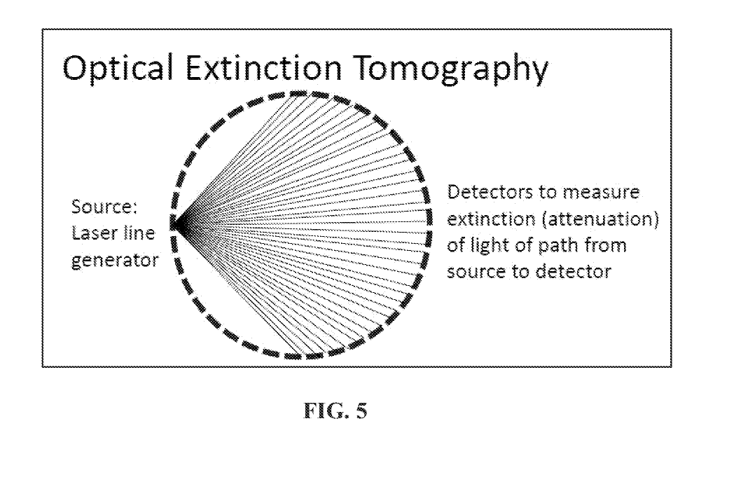

The sources and detectors are mounted onto the support frame (FIG. 5). Ideally, (along with any optics) would be flush mounted so as not to interfere with the flow being measured.

The support frame can be made of metal or other advanced structural materials.

### Light Sources

Since the purpose of the invention is to reconstruct particle density by measuring optical extinction, it is necessary to have bright sources distributed around the supporting frame. The extinction along rays from each light source is measured on the detectors. Typically, the light sources are lasers, the illumination of which can be precisely controlled by electrical signals. Although lasers are used in the preferred embodiment, other intense light sources could also be used. It may be desirable for the extinction model to use monochromatic light. In addition to the monochromatic nature of laser light, spectral filters can also be used.

In the present embodiment, it is desired to reconstruct particle density along a plane roughly transverse to the flow through the wind tunnel. Accordingly, it is desirable to focus the energy along an illumination half-plane. While this can be accomplished with collimation (as in U.S. Pat. No. 6,184,989, it is better accomplished with optical elements designed to focus the light energy onto a half plane and disperse the energy as evenly as possible among all directions from the source into the half-plane. In some embodiments, due to practical construction requirements, it may be necessary to shift the sources slightly off the plane of reconstruction. This will have the effect of reducing illumination onto detectors located close to the source. This should not have a major impact on reconstructed particle density.

### Detectors

The measurement of the invention is the measurement of the optical extinction along each path from the light sources to the detectors. The detectors are objects mounted along the support frame which are sensitive to light. It is desirable for these detectors to have wide collection angles, so a preferred embodiment will have optics surrounding each detector to widen the collection angle. While in the preferred embodiment the detectors on the ring will be fiber optic cables with collection angle widening optics, other detection options will also work. In the preferred embodiment, each end of the fiber optic cables opposite that collecting the light from the sources is mounted so as to point directly at a known location on a charge-coupled device (CCD) array. Thus, the light intensity on the detector can be measured by a CCD readout. As a practical consideration, the invention may have extra fibers not normally attached to the frame which can be used as spares to replace defective ones attached to the frame.

The detectors are nominally distributed on the support frame in the plane of illumination. They may be slightly off the desired plane of reconstruction if practical mounting constraints require it. In a nominal embodiment on a circular frame the detectors are evenly spaced and either completely or partially interlaced with the sources. The positions of the sources and detectors, together, determine the “geometry” of the acquisition. Other geometries can include features such as non-uniform spacing between either the sources, detectors, or both. Also possible are geometries where the sources and/or the detectors are slightly displaced from the measurement plane. Other geometries include evenly spaced detectors shifted by a fixed amount from the nominal interlaced geometry. One such embodiment is known as a quarter detector shift. Detectors may be placed outside of the direct source illumination plane in order to make direct measurements of scatter.

### Measurement Model

The measurement model has some similarity to that arising in medical Computed Tomography (CT). However, our application has several significant differences. In both medical applications and ours, the sources are in a ring outside the object and detectors are situated on a fan across from the source. However, in medical applications, the object of interest occupies a relatively small region about the center of the source ring. In the present application, the detectors are situated on the source ring, and the region of interest encompasses the entire interior of the cross-section, (though the central region here is also of primary importance).

The particle density distribution is computed by measuring the optical extinction along rays from the sources to the detectors. This data is reconstructed using tomographic algorithms to give a probability of extinction at a given location in the cross section. Using extinction models with expected particle size distributions, the material density can be recovered. In the present embodiment, the extinction model uses single particle scattering. If in other applications, a single particle scattering model is not sufficient, diffraction tomographic reconstruction techniques can be used instead of the Radon inversion methods used with the single scattering model.

To measure optical extinction with the preferred embodiment, three steps are needed. First, the acquisition CCD is calibrated by measuring the dark current. That is, with no sources illuminated, data are acquired. This gives a measure of the detection signal in the absence of stimulation, and allows the actual measurements to be calibrated. Typically, this does not need to be repeated frequently, as it is a characteristic of the measurement CCD camera.

Next, data are acquired with no flow or particles present. This gives the unextinguished light intensities. Finally, measurements are taken in the presence of particles. The ratio of intensities (after the dark current is subtracted) gives the extinction along the optical paths from each source to each detector. These extinction data along with the source and detector “geometry” are input to the tomographic reconstruction algorithms. Specifically, the model can include detector response characteristics related to (1) the incident angle of the source-detector line relative to the detector surface and (2) the source-detector distance.

While performing the flow absent measurement, various experimental anomalies can be detected. For example, defective sources and/or detectors can be identified. Also, detector gain levels can be calibrated to avoid saturation. Relative sensitivity profiles can be determined and exploited in reconstruction algorithms. Modifications to reconstruction algorithms to handle missing or unreliable data from known source-detector pairs can be incorporated. Anomalies (such as a part from the test section protruding into the measurement plane) during the flow-present acquisition can also be detected and handled.

### Acquisition

In order to perform an acquisition, the control circuitry pulses one source at a time (typically automatically under computer control). With the one source active, each detector measures the light intensity from that source along the connecting ray. In the preferred embodiment, this light is conducted to a specific region on a CCD in a high precision CCD camera. The charge on the sensor is read using the camera capabilities. The readout time from the CCD is the limiting factor in the timing of the source pulses. By custom design of camera readout protocols, the readout can be restricted only to the regions of the CCD onto which fibers have been connected. This significantly reduces readout time enabling a higher repetition rate.

The source pulse-readout sequence is repeated for each source on the support frame. After every source has been pulsed, sufficient data is available for the reconstruction engine to generate a particle density profile. The order in which each source is pulsed is referred to as the source pattern. Example patterns can be simply pulsing adjacent sources sequentially, or pulsing in a “star illumination pattern” and then sequentially using adjacent stars until all sources have been pulsed. For example, in an embodiment with 60 sources numbered sequentially from 1 to 60, one five point star would be sources 1, 25, 49, 13, 37. So the “star” illumination pattern would be 1, 25, 49, 13, 37, 2, 26, 50, 14, 38, 3, 27, 51, 15, 39, . . . .

The star illumination patterns are more robust with respect to time variations in the flow during acquisition. The illumination pattern to be used can be selected by the user and supported by the controlling hardware and software.

### Reconstruction

Tomographic reconstruction is the recovery of a quantity from a collection of line integrals of the quantity. The relevant quantity for this application is liquid water content. For the particle sizes expected in our embodiment and the optical path lengths across the measurement section, the extinction of a beam of light passing through the spray will be proportional to the line integral of liquid water content along the optical path.

For a single scattering model, the measurements can be converted to samples of the Radon transform of the extinction probability per unit length. In the present embodiment, novel methods, with some similarity to those used in commercial CT scanners, are used to recover the profile of the extinction probability per unit length (and hence the particle density). However, other methods specific to this application can also be used. For example, basis functions incorporating only low spatial frequencies can be used instead of the pixel based basis functions, as the expected particle densities do not have profiles with sharp edges for which high spatial frequencies are needed. Also, missing data can be handled by projection completion or interpolation. Alternatively, iterative reconstruction algorithms can also be applied. Note that some such algorithms which are not feasible in a medical setting are applicable here due to the reduced size of the data set.

Because the reconstruction region extends to the source ring, the standard reconstruction algorithms used in a medical setting must be modified to avoid significant artifacts. Another difference is that our sample density is much lower, so the available resolution in the reconstruction of the spray will be relatively low. On the other hand, since the spray itself is not expected to have sharp transitions, this is not expected to be a problem. Moreover, this a priori information can be exploited by modeling the spray as a superposition of low spatial frequency functions, such as Gaussian shaped blobs.

in addition, as a consequence of the implementations for some of the reconstruction methods discussed above, a method can be employed to provide almost real-time temporal updates. After data for a full image has been obtained, each time a source has been pulsed as part of the next acquisition, the data from that partial acquisition can replace the data from the previous pulsing of the same source. Only the new data needs to be processed to obtain an updated image. This idea can be applied to data obtained from any group of sources, such as a star in the star illumination patterns.

As an alternative to the traditional medical-type reconstruction, an algorithm has been developed which incorporates this a priori knowledge to reduce the computational complexity. It should be noted that the alternative algorithm does not scale well to the medical setting, but is well suited for use with the sampling densities available here. In this algorithm, the measurements are simulated for each possible Gaussian blob. The acquired spray measurement is fit to a linear combination of the simulated blob measurements. The corresponding linear combination of low spatial frequency functions is taken as the reconstructed image. This method is easily adapted to handle minor malfunctions in the acquisition system such as a dark source, or a dead detector.

In order to reduce streaking artifacts, the sampled data are up-sampled from 120×60 to 480×240. This up-sampling preserves the original bandwidth of the data. The reconstruction is based on this original bandwidth and does not improve the resolution, even though the reconstruction is performed on a finer grid. Although this method does well in the central ⅔ of the field of view, it is not as robust in the outer ring due to the uneven coverage of source-detector paths through the outer ring, and also because the filtered backprojection algorithm relies on an approximation which is less valid at reconstruction points close to the source ring.

For the rectangular frame as shown in FIG. 4, a different reconstruction method can be successfully used. The Truncated Singular Value Decomposition (TSVD) algorithm is a well-known method for regularizing the solution for the matrix equation, y=Rx.

In the context of the rectangular geometry, the column vector y holds the measured data, with each element corresponding to a source-detector pair. Each element of the column vector x corresponds to the cloud density at a position within the rectangular geometry. The matrix R implements the discretized Radon Transform which takes a cloud distribution x to the measurement y.

Because the linear equation is typically both over or under constrained, it is solved by use of the pseudo-inverse, R⁺, x=R⁺y, which gives the vector x with minimum norm that also minimizes the residual between R and Rx.

Unfortunately, when R⁻is ill-conditioned, meaning that some components in the data y will have a greatly magnified influence in the solution x, it is necessary to regularize R⁺as noise in the measurements (random, system, or numerical) will be amplified in the solution, swamping the reconstruction. Regularization effectively removes this inordinate amplification. The TSVD algorithm limits the acceptable magnification by ignoring the components in the data which would be unduly amplified in the solution. There is a trade-off between reconstruction resolution and fidelity and noise amplification which is tuneable by selection of a noise amplification threshold.

The application of the TSVD algorithm involves a time consuming computation of the SVD of the matrix R. This computation grows like the 4th power of the number of linear pixels in the reconstructed image. Fortunately, the SVD only needs to be computed once (offline), so it will not significantly impact cloud reconstructions.

### Data Analysis

Raw data and reconstructed density patterns are archived. The invention includes a module for analysis of reconstructed density patterns. In particular, temporal averaging of density patterns is available (and also available in real time).

### Simulator

The invention includes a simulator that has two (2) key capabilities. (1) Phantom object data is generated according to specified parameters. (2) Measurement data is produced using a phantom object and a model of the measurement system.

The phantom object can include cloud components or geometric components. Cloud components are used, for example, to characterize the fidelity provided by the measurement system and the reconstruction.

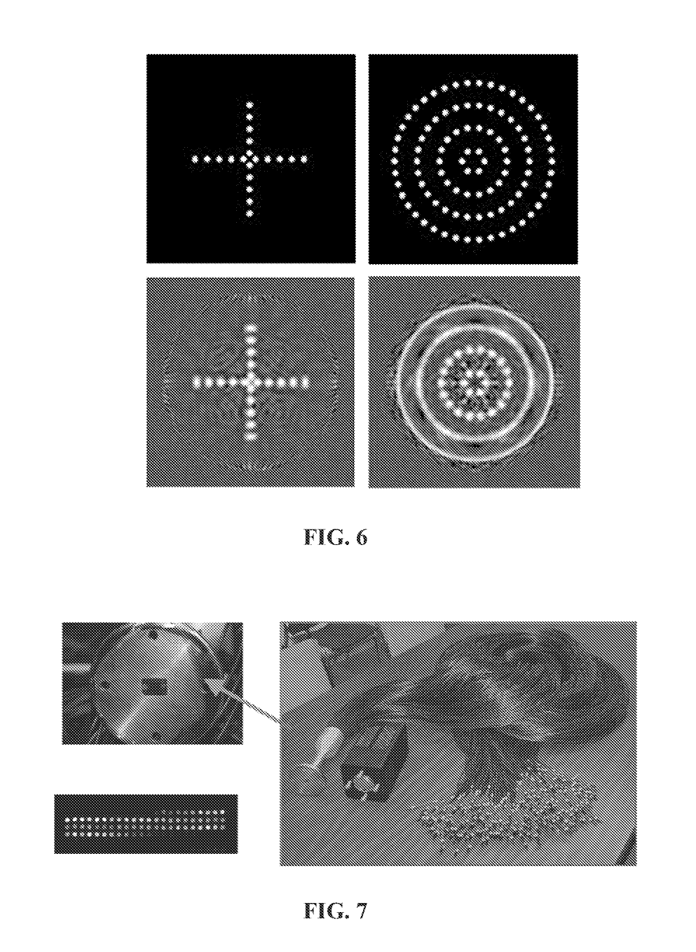

Geometric components are used, for example, to characterize the spatial resolution of the measurement system and the reconstruction. In FIG. 6, geometric simulation components are shown. In the cross image (upper left), the pins are placed 2-inches apart, thus there is a 1-inch gap between each pin. In the circles image (upper right), each ring is designed so that the circumferential gap is 1-inch between the 1-inch pins. The cross image will test radial resolution and the circles image will test angular resolution. The images in the lower left and lower right of FIG. 6 show the result of reconstructing the cross image and circle image using one embodiment of our reconstruction algorithm.

By specifying various parameters of the measurement system, the Simulator can be used to determine an effective hardware design. A key value of the Simulator is in the design of the reconstruction algorithm and parameters.

### Specific Embodiment Characteristics

In our circular embodiment, shown in FIG. 1 and described in REF. 2, the invention incorporates these specific characteristics.

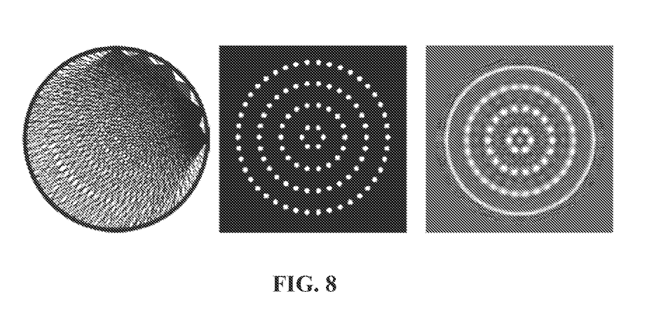

The light extinction tomography system consists of 60 equally spaced laser diodes with sheet generating optics and diffusing elements providing >300 degree coverage around the ring and 120 fiber optically coupled detection elements mounted every 3 degrees around a 36-inch. diameter ring. Photographs of this embodiment are shown in FIG. 2 and FIG. 3. Each detector utilizes a flashed opal input diffuser at the fiber entrance which is coupled to the CCD camera for simultaneous sampling of all 120 channels. The diffuser allows coupling of the laser light into the fibers at a very wide input angle of approximately +/−85 degrees with respect to the fiber face. The diffusers greatly increase the acceptance angle of the fibers at the cost of allowing only a small amount of the incident light to be coupled into the fiber. The laser diode sources are pulsed sequentially while the detectors acquire line-of-sight extinction data for each laser pulse. A custom timing/triggering circuit was built in-house and used to control the data acquisition. The optical fibers are direct coupled to the CCD through a fiberoptic faceplate. The imaged fibers are read out as a 5×5 pixel binned region of interest in the center of the fiber which yields a pixel per fiber or a 120 pixel image per sequential laser scan. The optical fiber and detection system are shown in FIG. 7.

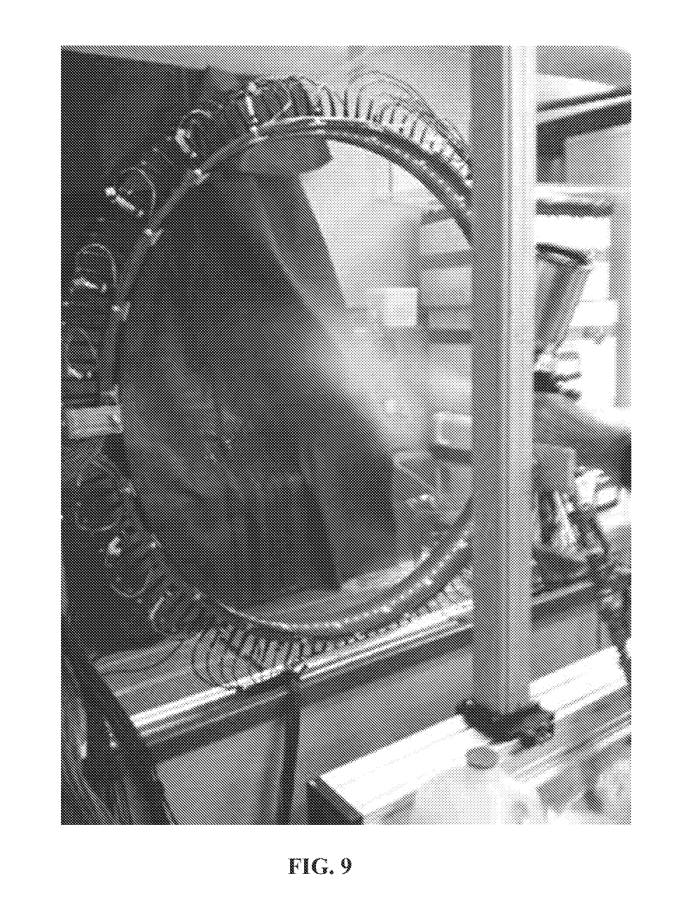

Using the computed tomography algorithms discussed in the previous section the extinction data is used to produce a plot of the relative water content in the measurement plane with spatial resolution better than 1 inch over the central 75% of the measurement area. FIG. 8 (left) illustrates the lines from several sources and the corresponding projections to the detectors. This gives some indication about the expected resolution as the area near the wall has a minimal amount of line crossing in multiple directions. A resolution study was performed to determine the expected resolution across the duct plane using simulated phantom data of 1 inch circles (FIG. 8 center image) and performing the reconstruction of the simulated line projection information. The reconstruction of the 60 source, 120 detector configuration is shown in the right image of FIG. 8 illustrating the loss of resolution with increasing radial distance from the center of the duct. The 1 inch circles are clearly evident in the inner two rings which represent approximately a 12 inch diameter. The third ring of circles from the center are now turned into ovals which shows a loss of angular resolution but each dot can still be recognized, this corresponds to a diameter of approximately 20 inches. The outer most dots are completely blended together at approximately a 30 inch diameter. This study was performed using a high spatial frequency model because of the abrupt high contrast of the dots on the black background. This high spatial resolution leads to reconstruction artifacts which can be ignored since the intent of the study is to confirm the expected resolution and not to minimize the reconstruction noise.

### Applications

The invention is a particle density detection system. Possible applications for this system are
1. Measure the density of ice or water particles inside a wind tunnel in real time and provide an archival record of particle density. In particular, spray patterns can be visualized. Anomalies in spray patterns (such as inoperative or malfunctioning nozzles or spray hot spots) can be detected. Can be used for engineering desired sprays.
2. As part of a wind tunnel instrumentation, could provide feedback and control for spray settings, both manually (human intervention) and automatic.
3. Measurement of a general spray system such as paint or water (FIG. 9) in industrial setting (also possibly with a control element).
4. Mounting the invention in the intake of a jet engine could provide real time and archival records of flight conditions. Upon detection of dangerous icing conditions, the system could alert the pilot and/or adjust engine parameters to ensure safe operation
5. Measure of atmospheric particulate density (such as volcanic ash).
6. Historical data can be compared for repeatability and to determine trends for sources, detectors and sprays, including individual nozzles.

### REFERENCES

1. Izen, S H, Bencic, T J, “Application of the Radon Transform to Calibration of the NASA-Glenn Icing Research Wind Tunnel,” Contemporary Mathematics, Vol. 278, 2001, pp. 147-166.
2. Bencic, T J, Fagan, A F, Van Zante, J F, Kirkegaard, J P, Rohler, D P, Maniyedath, A, Izen, S H, “Advanced Optical Diagnostics for Ice Crystal Cloud Measurements in the NASA Glenn Propulsion Systems Laboratory,” paper presented for American Institute of Aeronautics and Astronautics, AAIA Paper No. 2013-2678, 2013.

What is claimed is:

1. A light extinction tomography system as substantially described herein with reference to and as illustrated by the accompanying drawings.
